(19) United States
(12) Patent Application Publication
(10) Pub. No.: US 2025/0161190 A1
(43) Pub. Date: May 22, 2025

(54) ORAL CARE COMPOSITIONS

(71) Applicant: Colgate-Palmolive Company, New York, NY (US)

(72) Inventors: Rong DONG, Highland Park, NJ (US); Gregory SZEWCZYK, Flemington, NJ (US); Suman Kumar CHOPRA, Monroe, NJ (US); Santina Sally PERSICHETTI, Staten Island, NY (US)

(73) Assignee: Colgate-Palmolive Company, New York, NY (US)

(21) Appl. No.: 18/950,373

(22) Filed: Nov. 18, 2024

Related U.S. Application Data
(60) Provisional application No. 63/600,232, filed on Nov. 17, 2023.
(60) Provisional application No. 63/600,195, filed on Nov. 17, 2023.

(51) Int. Cl. A61K 8/81; A61K 8/21; A61K 8/25; A61Q 11/00

(52) U.S. Cl. CPC A61K 8/8147; A61K 8/21; A61K 8/25; A61Q 11/00; A61K 2800/28; A61K 2800/30; A61K 2800/48

(57) ABSTRACT

Oral care compositions; and specifically, oral care compositions providing enhanced stain resistance are disclosed herein. In accordance with an aspect of the invention, provided is an oral care composition including a thickening system comprising a cross-linked polyacrylic acid and/or a salt thereof, and one or more thickening agent selected from a polysaccharide, a thickening silica, and a combination of two or more thereof; a polyol; and from about 5 to about 64 wt. % of water, wherein all weight percentages are based on the total weight of the oral care composition.

### CROSS-REFERENCE TO RELATED APPLICATIONS

This application claims the benefit of priority from U.S. Patent Application No. 63/600,195, filed Nov. 17, 2023, titled Oral Care Compositions, and from U.S. Patent Application No. 63/600,232, filed Nov. 17, 2023, titled Oral Care Compositions, the content for both of which are hereby incorporated herein by reference in their entireties for all purposes.

### BACKGROUND

Residues of substances such as tobacco, tea, coffee, colored foods and the like in daily life are easy to deposit on tooth surfaces through saliva protein organic films on the surfaces of the teeth, if the teeth are not cleaned timely and effectively, the teeth can be gradually yellow or even black after long-time accumulation, exogenous stains and stains on the teeth are formed, and the oral hygiene and the tooth attractiveness are influenced. Whitening toothpastes typically aim at reducing exogenous staining of teeth, and can reduce stains on the surfaces of the teeth to a certain extent.

At present, consumers have strong demands on tooth whitening, and are interested in tooth whitening, so that some people whiten teeth by means of tooth washing and the like, but the cost is high, and certain risks exist. Most people want to whiten teeth on various toothpastes with whitening effects, the existing whitening toothpastes mostly adopt special granular abrasive agents or peroxides to be added into the toothpastes to achieve the whitening effect, but the granular abrasive agents can damage tooth enamel and damage the tooth surface, so that tooth stains are more easily deposited on the tooth surface.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to oral care compositions and, e.g., oral care compositions providing enhanced stain resistance. In accordance with an aspect of the invention, provided is an oral care composition including a thickening system comprising a cross-linked polyacrylic acid and/or a salt thereof, and one or more thickening agent selected from a polysaccharide, a thickening silica, and a combination of two or more thereof; a polyol; and from about 14 to about 64 wt. % of water, wherein all weight percentages are based on the total weight of the oral care composition.

In accordance with another aspect of the invention, provided is an oral care composition including a thickening system comprising: from about 0.2 to about 5 wt. % of a cross-linked polyacrylic acid and/or a salt thereof, and one or more thickening agent selected from a polysaccharide, a thickening silica, and a combination of two or more thereof; a polyol system comprising glycerin and sorbitol present in a weight ratio of glycerin to sorbitol of about 1:1 to about 3:1; and from about 5 to about 64 wt. % of water, wherein the oral care composition is free of whitening agents, and all weight percentages are based on the total weight of the oral care composition.

A list of non-limiting exemplary embodiments is provided below:

In accordance with an embodiment 1, provided is an oral care composition comprising: a thickening system comprising: a cross-linked polyacrylic acid and/or a salt thereof, and one or more thickening agent selected from a polysaccharide, a thickening silica, and a combination of two or more thereof; and from about 14 to about 64 wt. % of water, wherein all weight percentages are based on the total weight of the oral care composition.

In accordance with an embodiment 2, provided is the oral care composition according to embodiment 1, wherein the cross-linked polyacrylic acid and/or the salt thereof is a branched polyacrylic acid and/or a salt thereof.

In accordance with an embodiment 3, provided is the oral care composition according to embodiment 1 or embodiment 2, wherein the cross-linked polyacrylic acid and/or the salt thereof comprises a carbomer.

In accordance with an embodiment 4, provided is the oral care composition according to any foregoing embodiment, wherein the cross-linked polyacrylic acid and/or the salt thereof consist of one or more carbomer.

In accordance with an embodiment 5, provided is the oral care composition according to any foregoing embodiment, wherein the cross-linked polyacrylic acid and/or the salt thereof is present in an amount from about 0.2 to about 5 wt. %, optionally, from about 0.2 to about 1 wt. %.

In accordance with an embodiment 6, provided is the oral care composition according to any foregoing embodiment, further comprising one or more polyol, optionally, present in a total amount of about 20 to about 40 wt. %.

In accordance with an embodiment 7, provided is the oral care composition according to embodiment 6, wherein the one or more polyol is a polyol system.

In accordance with an embodiment 8, provided is the oral care composition according to embodiment 7, wherein the polyol system comprises at least two polyols selected from glycerin, inositol, maltitol, mannitol, sorbitol, xylitol, propylene glycol, polypropylene glycol (PPG), polyethylene glycol (PEG), a block copolymer of PPG and PEG, a saccharide, and a combination of two or more thereof.

In accordance with an embodiment 9, provided is the oral care composition according to embodiment 7 or embodiment 8, wherein the polyol system consists of at least two polyols selected from glycerin, inositol, maltitol, mannitol, sorbitol, xylitol, propylene glycol, polypropylene glycol (PPG), polyethylene glycol (PEG), and a combination of two or more thereof.

In accordance with an embodiment 10, provided is the oral care composition according to one of embodiment 6 to embodiment 9, wherein the polyol system comprises glycerin and sorbitol.

In accordance with an embodiment 11, provided is the oral care composition according to embodiment 10, wherein the glycerin and sorbitol are present in a weight ratio of the glycerin to the sorbitol of about 1:1 to about 3:1.

In accordance with an embodiment 12, provided is the oral care composition according to one of embodiment 6 to embodiment 11, wherein the total amount of polyol in the oral care composition is from about 20 to about 33 wt. %.

In accordance with an embodiment 13, provided is the oral care composition according to any foregoing embodiment, further comprising an abrasive, optionally, in an amount from about 1 to about 35 wt. %.

In accordance with an embodiment 14, provided is the oral care composition according to embodiment 13, wherein the abrasive is selected from silica, silicate, silicon, bentonite, zeolite, kaolin, mica, pumice, calcium carbonate, cuttlebone, insoluble phosphates, polycarbonate, microcrystalline wax, microcrystalline cellulose, and a combination of two or more thereof.

In accordance with an embodiment 15, provided is the oral care composition according to embodiment 13, wherein the abrasive consists of silica, silicate, silicon, bentonite, zeolite, kaolin, mica, pumice, calcium carbonate, cuttlebone, insoluble phosphates, polycarbonate, microcrystalline wax, microcrystalline cellulose, and a combination of two or more thereof.

In accordance with an embodiment 16, provided is the oral care composition according to one of embodiment 13 to embodiment 15, wherein the abrasive comprises silica.

In accordance with an embodiment 17, provided is the oral care composition according to any foregoing embodiment, wherein the water is present in an amount from about 30 to about 50 wt. %.

In accordance with an embodiment 18, provided is the oral care composition according to any foregoing embodiment, wherein the water is present in an amount from about 32 to about 45 wt. %.

In accordance with an embodiment 19, provided is the oral care composition according to any foregoing embodiment, further comprising a fluoride source, optionally, in an amount from about 0.1 to about 7 wt. %.

In accordance with an embodiment 20, provided is the oral care composition according to embodiment 19, wherein the fluoride source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

In accordance with an embodiment 21, provided is the oral care composition according to any foregoing embodiment, further comprising an anti-calculus agent in an amount from about 0.5 to about 10 wt. %.

In accordance with an embodiment 22, provided is the oral care composition according to embodiment 21, wherein the anti-calculus agent is selected from tetrasodium pyrophosphate, ammonium tripolyphosphate, hexametaphosphate salt, polycarboxylate polymers, polyvinyl methyl ether/maleic anhydride copolymers, and a combination of two or more thereof.

In accordance with an embodiment 23, provided is the oral care composition according to any foregoing embodiment, wherein the thickening system is present in an amount about 2 to about 15 wt. %.

In accordance with an embodiment 24, provided is the oral care composition according to any foregoing embodiment, wherein the thickening system comprises a thickening silica.

In accordance with an embodiment 25, provided is the oral care composition according to any foregoing embodiment, wherein the thickening system comprises a polysaccharide.

In accordance with an embodiment 26, provided is the oral care composition according to embodiment 25, wherein the polysaccharide is a natural gum.

In accordance with an embodiment 27, provided is the oral care composition according to embodiment 26, wherein the natural gum is selected from alginate, pectin, carrageenan, gum arabic, tragacanth gum, karaya gum, agarose, guar gum, pechtin, gellan gum, tara gum, locust bean gum, tamarind gum, locust bean gum, Seneca gum, sclerotium gum, gellan gum, xanthan gum, and a combination of two or more thereof.

In accordance with an embodiment 28, provided is the oral care composition according to any foregoing embodiment, wherein the thickening system comprises xanthan gum.

In accordance with an embodiment 29, provided is the oral care composition according to any foregoing embodiment, wherein the oral care composition is substantially free of or free of polyvinyl thickeners.

In accordance with an embodiment 30, provided is the oral care composition according to any foregoing embodiment, wherein the oral care composition is substantially free of or free of peroxides.

In accordance with an embodiment 31, provided is the oral care composition according to any foregoing embodiment, wherein the oral care composition is substantially free of or free of whitening agents.

In accordance with an embodiment 32, provided is an oral care composition comprising: a thickening system comprising: from about 0.2 to about 5 wt. % of a cross-linked polyacrylic acid and/or a salt thereof, and one or more thickening agent selected from a polysaccharide, a thickening silica, and a combination of two or more thereof; a polyol system comprising glycerin and sorbitol present in a weight ratio of glycerin to sorbitol of about 1:1 to about 3:1; and from about 5 to about 64 wt. % of water, wherein the oral care composition is free of whitening agents, and all weight percentages are based on the total weight of the oral care composition.

In accordance with an embodiment 33, provided is the oral care composition according to embodiment 32, wherein the cross-linked polyacrylic acid and/or a salt thereof is present in an amount from about 0.5 to about 4 wt. %.

In accordance with an embodiment 34, provided is the oral care composition according to embodiment 32 or embodiment 33, wherein a total amount of polyol in the oral care composition is from about 20 to about 40 wt. %.

In accordance with an embodiment 35, provided is the oral care composition according to embodiment 34, wherein the total amount of polyol in the oral care composition is from about 20 to about 33 wt. %.

In accordance with an embodiment 36, provided is the oral care composition according to one of embodiment 32 to embodiment 35, wherein the thickening system comprises a thickening silica.

In accordance with an embodiment 37, provided is the oral care composition according to one of embodiment 32 to embodiment 36, wherein the thickening system comprises a polysaccharide, the polysaccharide being a natural gum selected from alginate, pectin, carrageenan, gum arabic, tragacanth gum, karaya gum, agarose, guar gum, pechtin, gellan gum, tara gum, locust bean gum, tamarind gum, locust bean gum, Seneca gum, sclerotium gum, gellan gum, xanthan gum, and a combination of two or more thereof.

In accordance with an embodiment 38, provided is the oral care composition according to any foregoing embodiment, wherein the oral care composition is substantially free or free of calcium carbonate.

In accordance with an embodiment 39, provided is the oral care composition according to any foregoing embodiment, wherein the oral care composition has a relative dental abrasivity of about 10 to about 250, as determined pursuant to standards DIN EN ISO 11609.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition. The symbol “°” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, certain compounds may be characterized as both a polyol and a sweetener. If a particular oral care composition recites both a polyol and a sweetener, xylitol will serve only as either a polyol or a sweetener—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to oral care compositions and, e.g., oral care compositions providing enhanced stain resistance. While traditional whitening oral care compositions focus on the use of whitening agents to whiten teeth, the inventors surprisingly discovered that certain oral care compositions disclosed herein can reduce and/or prevent future stains by increasing the resistance of teeth to stains.

In accordance with an aspect of the invention, provided is an oral care composition including a thickening system comprising a cross-linked polyacrylic acid and/or a salt thereof, and one or more thickening agent selected from a polysaccharide, a thickening silica, and a combination of two or more thereof; a polyol; and from about 14 to about 64 wt. % of water, wherein all weight percentages are based on the total weight of the oral care composition.

In accordance with another aspect of the invention, provided is an oral care composition including a thickening system comprising: from about 0.2 to about 5 wt. % of a cross-linked polyacrylic acid and/or a salt thereof, and one or more thickening agent selected from a polysaccharide, a thickening silica, and a combination of two or more thereof; a polyol system comprising glycerin and sorbitol present in a weight ratio of glycerin to sorbitol of about 1:1 to about 3:1; and from about 14 to about 64 wt. % of water, wherein the oral care composition is free of whitening agents, and all weight percentages are based on the total weight of the oral care composition.

The oral care compositions may, in some preferred cases, have a reduced amount, be substantially free of, or free of polyvinyl thickeners. For example, the oral care composition may preferably have about 4 wt. % or less, about 3 wt. % or less, about 2 wt. % or less, about 1 wt. % or less, about 0.5 wt. % or less, or about 0.1 wt. % or less, based on the weight of the oral care composition, of polyvinyl thickeners. In at least one embodiment, the oral care composition comprises about 0 wt. % or 0 wt. % of polyvinyl thickeners.

Additionally and/or alternatively, the oral care compositions may, preferably, have a reduced amount, be substantially free of, or free of peroxides. For example, the oral care composition may preferably have about 4 wt. % or less, about 3 wt. % or less, about 2 wt. % or less, about 1 wt. % or less, about 0.5 wt. % or less, or about 0.1 wt. % or less, based on the weight of the oral care composition, of peroxides. In at least one embodiment, the oral care composition comprises about 0 wt. % or 0 wt. % of peroxides.

In certain preferred embodiments, the oral care compositions have a reduced amount, be substantially free of, or free of whitening agents. For example, the oral care composition may preferably have about 4 wt. % or less, about 3 wt. % or less, about 2 wt. % or less, about 1 wt. % or less, about 0.5 wt. % or less, or about 0.1 wt. % or less, based on the weight of the oral care composition, of whitening agents. In at least one embodiment, the oral care composition comprises about 0 wt. % or 0 wt. % of whitening agents.

Additionally and/or alternatively, the oral care compositions may preferably have a reduced amount, be substantially free of, or free of carbonates, such as calcium carbonate. For example, the oral care composition may preferably have about 4 wt. % or less, about 3 wt. % or less, about 2 wt. % or less, about 1 wt. % or less, about 0.5 wt. % or less, or about 0.1 wt. % or less, based on the weight of the oral care composition, of carbonates, such as calcium carbonate. In at least one embodiment, the oral care composition comprises about 0 wt. % or 0 wt. % of carbonates, such as calcium carbonate.

Preferably, the oral care composition may achieve the stain resistance benefits while simultaneously have a desirable relative dental abrasivity. In certain preferred embodiments, the oral care composition has a relative dental abrasivity of about 250 or less, about 200 or less, about 150 or less, about 125 or less, about 100 or less, or about 75 or less, as determined pursuant to standards DIN EN ISO 11609. Additionally or alternatively, the oral care composition may preferably have about 10 or more, about 50 or more, or about 75 or more, as determined pursuant to standards DIN EN ISO 11609. For example, the oral care composition may preferably have a relative dental abrasivity of about 10 to about 250, about 50 to about 200, or any other range formed from the disclosure herein, as determined pursuant to standards DIN EN ISO 11609.

The oral care compositions may be in the form of dentifrice, toothpaste, mouthwash, gels, varnishes, leave-on products (e.g., in the form of a gel, varnish, etc.), and/or the like. Suitable components, such as those listed below, may be included or excluded from the formulations for the oral care compositions depending on the specific combination of other ingredients and the form of the oral care compositions. Additionally or alternatively, the oral care compositions may in some embodiments have a single phase, which contains that components and/or ingredients of the oral care composition. In other embodiments, the oral care composition may include two or more phases, such as two, three, four, or five phases.

The oral care compositions typically comprise a thickening system comprising one or more cross-linked polyacrylic acid and/or a salt thereof(s) and one or more thickening agent selected from a polysaccharide, a thickening silica, and a combination of two or more thereof. In some embodiments, the thickening system consists of one or more cross-linked polyacrylic acid and/or a salt thereof(s) and one or more thickening agent selected from a polysaccharide, a thickening silica, and a combination of two or more thereof, such as those described herein.

The thickening system may be present in the oral care compositions in an amount ranging from about 2 to about 15 wt. %, based on the total weight of the oral care composition. For instance, total amount of the thickening system may, preferably, be from about 2 to about 10 wt. %, about 2 to about 8 wt. %, about 2 to about 7 wt. %, about 2 to about 6 wt. %, about 2 to about 5 wt. %, about 2 to about 4 wt. %; from about 3 to about 10 wt. %, about 3 to about 8 wt. %, about 3 to about 7 wt. %, about 3 to about 6 wt. %, about 3 to about 5 wt. %; from about 4 to about 15 wt. %, about 4 to about 12 wt. %, about 4 to about 10 wt. %, about 4 to about 8 wt. %, about 4 to about 7 wt. %, about 4 to about 6 wt. %, about 4 to about 5 wt. %; from about 5 to about 15 wt. %, about 5 to about 12 wt. %, about 5 to about 10 wt. %, about 5 to about 8 wt. %, about 5 to about 7 wt. %, about 5 to about 6 wt. %; from about 6 to about 10 wt. %, about 6 to about 8 wt. %; from about 7 to about 15 wt. %, about 7 to about 12 wt. %, about 7 to about 10 wt. %, about 7 to about 9 wt. %; from about 9 to about 15 wt. %, about 9 to about 12 wt. %, about 9 to about 11 wt. %; from about 11 to about 15 wt. %, about 11 to about 14 wt. %, about 13 to about 15 wt. %, or any range or subrange thereof, based on the total weight of the oral care composition.

The thickening system typically comprises one or more cross-linked acrylic acid and/or a salt thereof, e.g., present in an amount from about 0.2 to about 4 wt. %, based on the total weight of the oral care composition. In some instances, the one or more cross-linked acrylic acid and/or a salt may, preferably, be present in an amount from about 0.2 to about 3 wt. %, about 0.2 to about 2.5 wt. %, about 0.2 to about 2 wt. %, about 0.2 to about 1.5 wt. %, about 0.2 to about 1.25 wt. %, about 0.2 to about 1 wt. %, about 0.2 to about 0.75 wt. %, about 0.2 to about 0.5 wt. %; from about 0.35 to about 4 wt. %, about 0.35 to about 3 wt. %, about 0.35 to about 2.5 wt. %, about 0.35 to about 2 wt. %, about 0.35 to about 1.5 wt. %, about 0.35 to about 1.25 wt. %, about 0.35 to about 1 wt. %, about 0.35 to about 0.75 wt. %, about 0.35 to about 0.5 wt. %; from about 0.5 to about 4 wt. %, about 0.5 to about 3 wt. %, about 0.5 to about 2.5 wt. %, about 0.5 to about 2 wt. %, about 0.5 to about 1.5 wt. %, about 0.5 to about 1.25 wt. %, about 0.5 to about 1 wt. %, about 0.5 to about 0.75 wt. %; from about 0.75 to about 4 wt. %, about 0.75 to about 3 wt. %, about 0.75 to about 2.5 wt. %, about 0.75 to about 2 wt. %, about 0.75 to about 1.5 wt. %, about 0.75 to about 1.25 wt. %, about 0.75 to about 1 wt. %; from about 1 to about 4 wt. %, about 1 to about 3 wt. %, about 1 to about 2.5 wt. %, about 1 to about 2 wt. %, about 1 to about 1.5 wt. %, about 1 to about 1.25 wt. %; from about 1.5 to about 4 wt. %, about 1.5 to about 3 wt. %, about 1.5 to about 2.5 wt. %, about 1.5 to about 2 wt. %; from about 2 to about 4 wt. %, about 2 to about 3 wt. %, about 2 to about 2.5 wt. %; from about 2.5 to about 4 wt. %, about 2.5 to about 3 wt. %, about 3 to about 4 wt. %, or any range or subrange thereof, based on the total weight of the oral care composition.

The cross-linked polyacrylic acid and/or a salt thereof may be selected from branched polyacrylic acids and/or salts thereof. In some instances, the polyacrylic acid and/or a salt thereof consists of branched polyacrylic acids and/or salts thereof. In further instances, however, the oral care composition includes at least one linear polyacrylic acid and/or a salt thereof.

The cross-linked polyacrylic acid and/or a salt thereof may be a carbomer. The carbomer may comprise or be selected from the group consisting of Carbopols. For instance, the personal care composition may include, or in some cases exclude, a polyacrylic acid and/or a salt thereof selected from Carbopol 2984, Carbopol 934, Carbopol Ultrez 10, Carbopol 940, Carbopol 941, Carbopol 956 Carbopol 981, Carbopol 2020, and a combination of two or more thereof. In some cases, the polyacrylic acid and/or a salt thereof comprises one or more of or is selected from the group consisting from Carbopol 2984, Carbopol 910, Carbopol 934P, Carbopol Ultrez 10, Carbopol 940, Carbopol 941, Carbopol 956, Carbopol 971P, Carbopol 974P, Carbopol 981, Carbopol 2020, and/or a combination of two or more thereof. In at least one embodiment, the carbomer comprises or consists of carbopol ETD NF 2020. Additionally or alternatively, the cross-linked polyacrylic acid and/or a salt thereof may be selected from carbomers.

The one or more thickening agent selected from a polysaccharide, a thickening silica, and a combination of two or more thereof may be present in an amount, preferably, from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The polysaccharide may be selected from celluloses, cellulose derivatives, natural gum, and combinations thereof. In some embodiments, the polysaccharide is a natural gum. Examples of natural gums that may be included in, or in certain embodiments excluded from, the oral care composition include alginate, pectin, carrageenan, gum arabic, tragacanth gum, karaya gum, agarose, guar gum, pechtin, gellan gum, tara gum, locust bean gum, tamarind gum, locust bean gum, Seneca gum, sclerotium gum, gellan gum, xanthan gum, or a combination of two or more thereof. For instance, the thickening system may include a natural gum comprising gum arabic, tragacanth gum, karaya gum, agarose, guar gum, pechtin, gellan gum, tara gum, locust bean gum, tamarind gum, locust bean gum, Seneca gum, sclerotium gum, gellan gum, xanthan gum, or a combination thereof. In some cases, the oral care composition includes a natural gum selected from xanthan gum, carrageenan, pechtin, alginate, pectin, and a combination of two or more thereof. In at least one embodiment, the natural gum comprises xanthan gum.

The polysaccharide may be a cellulose. Examples of celluloses include cellulose ether chosen from carboxymethylcellulose (CMC), ethylcellulose (EC), hydroxypropylcellulose (HPC), hydroxypropylmethylcellulose (HPMC), methylhydroxyethylcellulose (MHEC), and mixtures thereof. In some embodiments, the cellulose is a modified cellulose selected from microcrystalline cellulose (MCC), e.g., in the anhydrous or hydrated forms thereof, and cellulose ethers such as hydroxypropylcellulose (HPC) and hydroxypropylmethylcellulose (HPMC).

The thickening system may include a thickening silica. The thickening silica, sometimes referred to as silica thickeners, which form polymeric structures or gels in aqueous media, may be present. The thickening silica is typically physically and functionally distinct from the particulate silica abrasives as the silica thickeners are very finely divided and provide little or no abrasive action.

The thickening system may include a thickening silica having a particle size distribution (D95) less than or equal to 7 μm and a BET surface area of greater than or equal to 150 m²/g. In some cases, the thickening silica has a BET surface area of greater than or equal to 175 m²/g, greater than or equal to 200 m²/g, or greater than or equal to 250 m²/g. For example, the thickening silica may have a BET surface area of about 150 to about 350 m²/g, about 170 to about 350 m²/g, about 190 to about 350 m²/g, about 210 to about 350 m²/g, about 230 to about 350 m²/g, about 250 to about 350 m²/g, about 270 to about 350 m²/g, about 290 to about 350 m²/g, about 310 to about 350 m²/g, about 330 to about 350 m²/g; from about 150 to about 300 m²/g, about 170 to about 300 m²/g, about 190 to about 300 m²/g, about 210 to about 300 m²/g, about 230 to about 300 m²/g, about 250 to about 300 m²/g, about 270 to about 300 m²/g; from about 150 to about 275 m²/g, about 170 to about 275 m²/g, about 190 to about 275 m²/g, about 210 to about 275 m²/g, about 230 to about 275 m²/g, about 250 to about 275 m²/g; from about 150 to about 250 m²/g, about 170 to about 250 m²/g, about 190 to about 250 m²/g, about 210 to about 250 m²/g, about 230 to about 250 m²/g; from about 150 to about 225 m²/g, about 170 to about 225 m²/g, about 190 to about 225 m²/g, about 210 to about 225 m²/g, or any range or subrange thereof.

Additionally or alternatively, the thickening silica may have an oil absorption of greater than or equal to about 200 mL/100 g, about 240 mL/100 g, about 250 mL/100 g, or any range thereof. For instance, the thickening silica may have an oil absorption of about 200 to about 250 mL/100 g, about 210 to about 250 mL/100 g, about 220 to about 250 mL/100 g, about 230 to about 250 mL/100 g; from about 200 to about 240 mL/100 g, about 210 to about 240 mL/100 g, about 220 to about 240 mL/100 g, about 230 to about 240 mL/100 g; from about 200 to about 230 mL/100 g, about 210 to about 230 mL/100 g, about 220 to about 230 mL/100 g, or any range or subrange thereof.

Other thickening agent, which may in some cases be included or excluded from the oral care composition, include acrylates, polyvinyl pyrrolidone, polyitaconates, and acrylamides. Polyvinyl pyrrolidone generally refers to a polymer containing vinylpyrrolidone (e.g., N-vinylpyrrolidone, N-vinyl-2-pyrrolidione, and N-vinyl-2-pyrrolidinone) as a monomeric unit. The monomeric unit may include a polar imide group, four non-polar methylene groups, and a non-polar methane group. The acrylates may be diacrylates and/or methacrylates. Examples of acrylates include, e.g., isobutyl acrylate, tert-butyl acrylate, 2-ethylhexyl acrylate, lauryl acrylate, lauryl/tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-ethoxyethoxyethyl acrylate, 2-phenoxyethyl acrylate, tetrahydrofurfuryl acrylate, 2-hydroxyethyl acrylate, 2-hydroxypropyl acrylate, 4-hydroxybutyl acrylate, dimethylaminoethyl acrylate, 1,4-butanediol acrylate, or a combination of two or more thereof. Examples of acrylamides include, but are not limited to, acrylamide, methacrylamide and di(C₁-C₃₀) alkyl-acrylamides and -methacrylamides, such as those of methyl, ethyl, propyl, butyl, pentyl, hexyl and the like.

The oral care compositions may include one or more polyol(s). In some cases, the oral care composition includes a polyol system comprising a plurality of polyols. The total amount of polyols present in the oral care composition about 20 to about 40 wt. %, about 20 to about 37 wt. %, about 20 to about 35 wt. %, about 20 to about 33 wt. %, about 20 to about 31 wt. %, about 20 to about 29 wt. %, about 20 to about 27 wt. %, about 20 to about 25 wt. %; from about 24 to about 40 wt. %, about 24 to about 37 wt. %, about 24 to about 35 wt. %, about 24 to about 33 wt. %, about 24 to about 31 wt. %, about 24 to about 29 wt. %; from about 28 to about 40 wt. %, about 28 to about 37 wt. %, about 28 to about 35 wt. %, about 28 to about 33 wt. %, about 28 to about 31 wt. %; from about 30 to about 40 wt. %, about 30 to about 37 wt. %, about 30 to about 35 wt. %, about 30 to about 33 wt. %; from about 32 to about 40 wt. %, about 32 to about 37 wt. %, about 32 to about 35 wt. %; from about 34 to about 40 wt. %, about 34 to about 37 wt. %, about 36 to about 40 wt. %, about 36 to about 38 wt. %, or any range or subrange thereof, based on the total weight of the oral care composition.

In some cases, the amount of each polyol in the oral care composition may be from about 5 to about 35 wt. %, about 5 to about 30 wt. %, about 5 to about 25 wt. %, about 5 to about 21 wt. %, about 5 to about 17 wt. %, about 5 to about 14 wt. %, about 5 to about 11 wt. %; from about 10 to about 35 wt. %, about 10 to about 30 wt. %, about 10 to about 25 wt. %, about 10 to about 21 wt. %, about 10 to about 17 wt. %, about 10 to about 14 wt. %; from about 15 to about 35 wt. %, about 15 to about 30 wt. %, about 15 to about 25 wt. %, about 15 to about 21 wt. %, about 15 to about 19 wt. %; from about 18 to about 35 wt. %, about 18 to about 30 wt. %, about 18 to about 25 wt. %, about 18 to about 21 wt. %; from about 21 to about 35 wt. %, about 21 to about 30 wt. %, about 21 to about 25 wt. %; from about 24 to about 35 wt. %, about 24 to about 30 wt. %; from about 27 to about 35 wt. %, about 27 to about 30 wt. %, or any range or subrange thereof, based on the total weight of the oral care composition.

The polyol(s) may be chosen from glycols or compounds with numerous hydroxyl groups. The one or more polyols may be liquid at ambient temperature (25° C.). In some preferred embodiments, the polyol(s) comprises glycerin, glycol, inositol, maltitol, mannitol, sorbitol, xylitol, propylene glycol, polypropylene glycol (PPG), polyethylene glycol (PEG), a block copolymer of PPG and PEG, a saccharide (e.g., fructose, glucose, sucrose and mixtures of saccharides, such as honey), or a combination of two or more thereof. For instance, the oral care composition may comprise maltitol, mannitol, sorbitol, xylitol, a polypropylene glycol (PPG), a polyethylene glycol (PEG), a block copolymer of PPG and PEG, or a combination or two or more thereof. The polyol may be a humectant including, e.g., glycol, sorbitol, glycerin, inositol, maltitol, mannitol, xylitol, propylene glycol, polypropylene glycol (PPG), polyethylene glycol (PEG), a saccharide, polyoxypropylene-polyoxyethylene block copolymer, and a combination of two or more thereof.

In some cases, the oral care composition includes one or more polyol selected from the group consisting of C₂-C₃₂ polyols. The one or more polyols may have from 2 to 32 carbon atoms, from 3 to 16 carbon atoms, or from 3 to 12 carbon atoms. For example, the oral care composition may comprise ethylene glycol, propylene glycol, butylene glycol, hexylene glycol, glycerin, diglycerin, diethylene glycol, and dipropylene glycol, or a combination of two or more thereof. Additional, non-limiting examples of polyols that may, optionally, be included in the oral care include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture of two or more thereof.

Additionally or alternatively, the oral care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof. In some embodiments, the polyol is a polyethylene glycol, such as polyethylene glycol 600. In some embodiments, the oral care composition comprises one or more polyethylene glycols, e.g., polyethylene glycols in a molecular weight range from 200 to 800. For example, the compositions may comprise one or more of polyethylene glycol 200, polyethylene glycol 300, polyethylene glycol 400, polyethylene glycol, 600 or polyethylene glycol 800.

In certain embodiments, the oral care composition comprises two or more polyols. In some preferred instances, at least one of the polyols is selected from or consists of glycerin and sorbitol. In at least one preferred embodiment, the oral care composition comprises glycerin and sorbitol. The oral care compositions may, in some preferred embodiments, have a polyol system comprising glycerin and sorbitol present in a weight ratio of glycerin to sorbitol of about 1:1 to about 3:1. For instance, the oral care composition may, preferably, have a weight ratio of glycerin to sorbitol of about 1:1 to about 3:1, about 1:1 to about 2.75:1, about 1:1 to about 2.5:1, about 1:1 to about 2.25:1, about 1:1 to about 2:1; from about 1:1 to about 3:1, about 1:1 to about 2.75:1, about 1:1 to about 2.5:1, about 1:1 to about 2.25:1, about 1:1 to about 2:1; from about 1.25:1 to about 3:1, about 1.25:1 to about 2.75:1, about 1.25:1 to about 2.5:1, about 1.25:1 to about 2.25:1, about 1.25:1 to about 2:1; from about 1.5:1 to about 3:1, about 1.5:1 to about 2.75:1, about 1.5:1 to about 2.5:1, about 1.5:1 to about 2.25:1, about 1.5:1 to about 2:1; from about 1.75:1 to about 3:1, about 1.75:1 to about 2.75:1, about 1.75:1 to about 2.5:1, about 1.75:1 to about 2.25:1, about 1.75:1 to about 2:1, or a range or subrange thereof.

The oral care compositions may include one or more abrasive(s), e.g., in an amount from about 1 to about 35 wt. %, based on the total weight of the oral care composition. In some embodiments, the one or more abrasive(s) is present in an amount from about 1 to about 30 wt. %, about 1 to about 25 wt. %, about 1 to about 21 wt. %, about 1 to about 17 wt. %, about 1 to about 14 wt. %, about 1 to about 11 wt. %; from about 5 to about 30 wt. %, about 5 to about 25 wt. %, about 5 to about 21 wt. %, about 5 to about 17 wt. %, about 5 to about 14 wt. %, about 5 to about 11 wt. %; from about 10 to about 35 wt. %, about 10 to about 30 wt. %, about 10 to about 25 wt. %, about 10 to about 21 wt. %, about 10 to about 17 wt. %, about 10 to about 14 wt. %; from about 15 to about 35 wt. %, about 15 to about 30 wt. %, about 15 to about 25 wt. %, about 15 to about 21 wt. %, about 15 to about 19 wt. %; from about 18 to about 35 wt. %, about 18 to about 30 wt. %, about 18 to about 25 wt. %, about 18 to about 21 wt. %; from about 21 to about 35 wt. %, about 21 to about 30 wt. %, about 21 to about 25 wt. %; from about 24 to about 35 wt. %, about 24 to about 30 wt. %; from about 27 to about 35 wt. %, about 27 to about 30 wt. %, or any range or subrange thereof, based on the total weight of the oral care composition.

The one or more abrasive(s) may include: silica, silicate, silicon, alumina (including calcined aluminum oxide), aluminosilicates, such as bentonite, zeolite, kaolin, and mica, siliceous or diatomaceous earth, pumice, calcium carbonate, cuttlebone, insoluble phosphates, composite resins, such as melamine resin, phenolic resin, and urea-formaldehyde resin, polycarbonate, silicon carbide, boron carbide, microcrystalline wax, microcrystalline cellulose, including combinations of colloidal microcrystalline cellulose and carboxymethylcellulose, and combinations and derivatives thereof. For example, the one or more abrasive may be selected from silicas including gels and precipitates, calcium carbonate, dicalcium orthophosphate dihydrate, calcium pyrophosphate, tricalcium phosphate, calcium polymetaphosphate, insoluble sodium polymetaphosphate, hydrated alumina, resinous abrasive materials such as particulate condensation products of urea and formaldehyde, and combinations thereof.

As used herein, “mica” refers to any of a group of hydrous aluminum silicate minerals with plate morphology and/or perfect basal (micaceous) cleavage. Mica can be, for example, sheet mica, scrap mica or flake mica, as exemplified by muscovite, biotite or phlogopite type micas. The abrasive may be selected from insoluble phosphates, such as orthophosphates, polymetaphosphates, pyrophosphates, and a combination thereof. Synthetic silicas include both silica gels and precipitated silicas that are prepared by the neutralization of aqueous silicate solutions with a strong mineral acid. Abrasives comprising silica may be useful in certain embodiments of the oral care composition. In certain embodiments, the oral compositions may comprise a particularly efficacious combination of silica abrasive particle species. For example, the abrasive(s) be selected from high cleaning silica.

The oral care compositions may include an abrasive system comprising two or more abrasives. For example, an abrasive system comprise 2 to 7, 2 to 6, 2 to 5, 2 to 5, or 2 to 4; 3 to 7, 3 to 6, 2 to 5, or 3 to 5 abrasives. The abrasives may comprise one or more cleaning abrasive and/or one or more polishing abrasives. As appreciated by one of skill in the art, a single abrasive species typically performs at least some cleaning and polishing simultaneously. However, particles are generally categorized in the art by the predominant effect they have on a target oral surface. Typically, “polishing abrasives” are considered to be relatively small particles having high hardness, where abrasives with relatively large particle sizes and low hardness are considered to be “cleaning abrasives.” In certain embodiments, the oral care composition comprises two or more abrasives comprising silica. In some embodiments, the first abrasive is selected to be a harder and smaller abrasive, e.g., a higher cleaning and/or polishing abrasive, and the second abrasive is a typical cleaning abrasive. In some embodiments, the oral care composition includes at least one polishing abrasive and/or at least one cleaning abrasive. Further examples of abrasives are disclosed in U.S. Patent Publication No. 2007/140986, which is incorporated herein in its entirety by reference for all purposes.

In some cases, the abrasive is selected from silica, silicate, silicon, bentonite, zeolite, kaolin, mica, pumice, calcium carbonate, cuttlebone, insoluble phosphates, polycarbonate, microcrystalline wax, microcrystalline cellulose, and a combination of two or more thereof. The oral care composition may have abrasive(s) consisting of silica, silicate, silicon, bentonite, zeolite, kaolin, mica, pumice, calcium carbonate, cuttlebone, insoluble phosphates, polycarbonate, microcrystalline wax, microcrystalline cellulose, and a combination of two or more thereof in certain embodiments. In at least one embodiment, the abrasive is a silica abrasive, such as a high cleaning silica abrasive.

The oral care compositions typically comprise an amount of water. The amount of water present in the oral care compositions may be about 5 to about 64 wt. % of water, based on the total weight of the oral care composition. For example, the amount of water in the oral care composition may be from about 5 to about 60 wt. %, about 5 to about 56 wt. %, about 5 to about 53 wt. %, about 5 to about 50 wt. %, about 5 to about 47 wt. %, about 5 to about 45 wt. %, about 5 to about 43 wt. %, about 5 to about 41 wt. %, about 5 to about 39 wt. %, about 5 to about 37 wt. %, about 5 to about 35 wt. %, about 5 to about 33 wt. %; from about 10 to about 60 wt. %, about 10 to about 56 wt. %, about 10 to about 53 wt. %, about 10 to about 50 wt. %, about 10 to about 47 wt. %, about 10 to about 45 wt. %, about 10 to about 43 wt. %, about 10 to about 41 wt. %, about 10 to about 39 wt. %, about 10 to about 37 wt. %, about 10 to about 35 wt. %, about 10 to about 33 wt. %; from about 14 to about 64 wt. %, about 14 to about 60 wt. %, about 14 to about 56 wt. %, about 14 to about 53 wt. %, about 14 to about 50 wt. %, about 14 to about 47 wt. %, about 14 to about 45 wt. %, about 14 to about 43 wt. %, about 14 to about 41 wt. %, about 14 to about 39 wt. %, about 14 to about 37 wt. %, about 14 to about 35 wt. %, about 14 to about 33 wt. %; from about 19 to about 64 wt. %, about 19 to about 60 wt. %, about 19 to about 56 wt. %, about 19 to about 53 wt. %, about 19 to about 50 wt. %, about 19 to about 47 wt. %, about 19 to about 45 wt. %, about 19 to about 43 wt. %, about 19 to about 41 wt. %, about 19 to about 39 wt. %, about 19 to about 37 wt. %, about 19 to about 35 wt. %, about 19 to about 33 wt. %; from about 24 to about 64 wt. %, about 24 to about 60 wt. %, about 24 to about 56 wt. %, about 24 to about 53 wt. %, about 24 to about 50 wt. %, about 24 to about 47 wt. %, about 24 to about 45 wt. %, about 24 to about 43 wt. %, about 24 to about 41 wt. %, about 24 to about 39 wt. %, about 24 to about 37 wt. %, about 24 to about 35 wt. %, about 24 to about 33 wt. %; from about 29 to about 64 wt. %, about 29 to about 60 wt. %, about 29 to about 56 wt. %, about 29 to about 53 wt. %, about 29 to about 50 wt. %, about 29 to about 47 wt. %, about 29 to about 45 wt. %, about 29 to about 43 wt. %, about 29 to about 41 wt. %, about 29 to about 39 wt. %, about 29 to about 37 wt. %; from about 32 to about 64 wt. %, about 32 to about 60 wt. %, about 32 to about 56 wt. %, about 32 to about 53 wt. %, about 32 to about 50 wt. %, about 32 to about 47 wt. %, about 32 to about 45 wt. %, about 32 to about 43 wt. %, about 32 to about 41 wt. %, about 32 to about 39 wt. %; from about 34 to about 64 wt. %, about 34 to about 60 wt. %, about 34 to about 56 wt. %, about 34 to about 53 wt. %, about 34 to about 50 wt. %, about 34 to about 47 wt. %, about 34 to about 45 wt. %, about 34 to about 43 wt. %, about 34 to about 41 wt. %; from about 39 to about 64 wt. %, about 39 to about 60 wt. %, about 39 to about 56 wt. %, about 39 to about 53 wt. %, about 39 to about 50 wt. %, about 39 to about 47 wt. %, about 39 to about 45 wt. %, about 39 to about 43 wt. %; from about 42 to about 64 wt. %, about 42 to about 60 wt. %, about 42 to about 56 wt. %, about 42 to about 53 wt. %, about 42 to about 50 wt. %, about 42 to about 47 wt. %; from about 45 to about 64 wt. %, about 45 to about 60 wt. %, about 45 to about 56 wt. %, about 45 to about 53 wt. %, about 45 to about 50 wt. %; from about 48 to about 64 wt. %, about 48 to about 60 wt. %, about 48 to about 56 wt. %, about 48 to about 53 wt. %; from about 54 to about 64 wt. %, about 54 to about 60 wt. %, or any range or subrange thereof, based on the total weight of the oral care composition.

The oral care compositions may comprise one or more surfactant(s) in an amount that may vary, but typically may be from about 0.1 to about 8 wt. %, based on the total weight of the oral care composition. For example, the amount of one or more surfactant(s) present in the oral care composition may be from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.3 to about 8 wt. %, about 0.3 to about 6 wt. %, about 0.3 to about 5 wt. %, about 0.3 to about 4 wt. %, about 0.3 to about 3 wt. %, about 0.3 to about 2 wt. %; from about 0.6 to about 8 wt. %, about 0.6 to about 6 wt. %, about 0.6 to about 5 wt. %, about 0.6 to about 4 wt. %, about 0.6 to about 3 wt. %, about 0.6 to about 2 wt. %; from about 0.9 to about 8 wt. %, about 0.9 to about 6 wt. %, about 0.9 to about 5 wt. %, about 0.9 to about 4 wt. %, about 0.9 to about 3 wt. %, about 0.9 to about 2 wt. %; from about 1.2 to about 8 wt. %, about 1.2 to about 6 wt. %, about 1.2 to about 5 wt. %, about 1.2 to about 4 wt. %, about 1.2 to about 3 wt. %, about 1.2 to about 2 wt. %; from about 1.5 to about 8 wt. %, about 1.5 to about 6 wt. %, about 1.5 to about 5 wt. %, about 1.5 to about 4 wt. %, about 1.5 to about 3 wt. %; from about 2 to about 8 wt. %, about 2 to about 6 wt. %, about 2 to about 5 wt. %, about 2 to about 4 wt. %, about 2 to about 3 wt. %; from about 3 to about 8 wt. %, about 3 to about 6 wt. %, about 3 to about 5 wt. %; from about 4 to about 8 wt. %, about 4 to about 6 wt. %, about 4 to about 5 wt. %, or any range or subrange thereof, based on the total weight of the oral care composition.

The one or more surfactant(s) may comprise an anionic surfactant, such as those selected from water-soluble, water-miscible salts of alkyl sulfate, such as those having from 8 to 20 carbon atoms in the alkyl radical (e.g., sodium alkyl sulfate) and the water-soluble or water-miscible salts of sulfonated monoglycerides of fatty acids having from 8 to 20 carbon atoms. Examples of anionic surfactants, which may be included or excluded in some instances from the oral care composition, include sodium lauryl sulfate, sodium lauryl sarcosinate, sodium cocoyl methyl taurate, sodium monoglyceride sulfate, sodium cetaryl sulfate, potassium cocoyl glycinate, sodium lauryl phosphate, sodium lauryl lactylate, sodium lauryl sulfoacetate, sodium lauryl glutamate, sodium lauryl isethionate, sodium laureth carboxylate, sodium dodecyl benzenesulfonate, and combinations thereof. In one embodiment, sodium lauryl sulfate is a preferred surfactant. Additional anionic surfactants are disclosed in U.S. Pat. No. 3,959,458, which is incorporated herein in its entirety for all purposes.

Further anionic surfactants of mention include long chain alkyl (C₆-C₂₂) materials, such as long chain alkyl sulfates, long chain alkyl sulfonates, long chain alkyl phosphates, long chain alkyl ether sulfates, long chain alkyl alpha olefin sulfonates, long chain alkyl taurates, long chain alkyl isethionates (SCI), long chain alkyl glyceryl ether sulfonates (AGES), sulfosuccinates and the like. These anionic surfactants can be alkoxylated, e.g., ethoxylated, although alkoxylation is not required.

The oral care composition may include a surfactant other than an anionic surfactant, such as a nonionic surfactant, amphoteric surfactant, or cationic surfactant. Examples of amphoteric surfactants include betaine surfactants, such as cocamidopropyl betaine, lauryl dimethyl betaine (sold under the trade name Macat LB), cetyl dimethyl betaine, and cocoamphodiacetate. Examples of nonionic surfactants, which may be included or excluded from the oral are composition, include glucosides, compounds produced by the condensation of alkylene oxide groups (hydrophilic in nature) with an organic hydrophobic compound, which may be aliphatic or alkylaromatic in nature. In some preferred embodiments, the oral care composition may be substantially free of or free of amphoteric surfactants.

The one or more nonionic surfactant(s) may be selected from glucosides, compounds produced by the condensation of alkylene oxide groups (hydrophilic in nature) with an organic hydrophobic compound, which may be aliphatic or alkylaromatic in nature. Examples of glucoside surfactants include decyl glucoside, stearyl glucoside, lauryl glucoside, coco-glucoside, cetearyl glucoside, decyl lauryl glucoside, lauroyl ethyl glucoside, myristoyl ethyl glucoside, oleoyl ethyl glucoside, or a combination of two or more thereof. In some preferred embodiments, the oral care composition may be substantially free of or free of nonionic surfactants. In at least one preferred embodiment, the oral care composition may be substantially free of or free of cationic surfactants.

In some embodiments, the oral care compositions may include one or more sweeteners. The oral care composition may include caloric sweeteners and/or non-caloric sweeteners. Examples of non-caloric sweeteners include saccharin, e.g., sodium saccharin, acesulfame, neotame, cyclamate or sucralose; natural high-intensity sweeteners, such as thaumatin, stevioside or glycyrrhizin; or sugar alcohols, such as sorbitol, xylitol, maltitol and mannitol. Examples of caloric sweeteners include sugars, such as fructose, glucose, sucrose, and high fructose syrups.

The one or more sweetener(s) may be present in the oral care composition in an amount from about 0.1 to about 50 wt. %, based on the total weight of the oral care composition. For example, the oral care composition may have a total amount of sweetener(s) from about 0.1 to about 40 wt. %, about 0.1 to about 30 wt. %, about 0.1 to about 20 wt. %, about 0.1 to about 10 wt. %, about 0.1 to about 5 wt. %, about 0.1 to about 3 wt. %; from about 1 to about 50 wt. %, about 1 to about 40 wt. %, about 1 to about 30 wt. %, about 1 to about 20 wt. %, about 1 to about 10 wt. %, about 1 to about 5 wt. %, about 1 to about 3 wt. %; from about 5 to about 50 wt. %, about 5 to about 40 wt. %, about 5 to about 30 wt. %, about 5 to about 20 wt. %, about 5 to about 10 wt. %; from about 10 to about 50 wt. %, about 10 to about 45 wt. %, about 10 to about 40 wt. %, about 10 to about 35 wt. %, about 10 to about 30 wt. %, about 10 to about 25 wt. %; from about 15 to about 50 wt. %, about 15 to about 45 wt. %, about 15 to about 40 wt. %, about 15 to about 37 wt. %, about 15 to about 34 wt. %, about 15 to about 31 wt. %, about 15 to about 28 wt. %, about 15 to about 25 wt. %; from about 20 to about 50 wt. %, about 20 to about 45 wt. %, about 20 to about 40 wt. %, about 20 to about 37 wt. %, about 20 to about 34 wt. %, about 20 to about 31 wt. %, about 20 to about 28 wt. %; from about 25 to about 50 wt. %, about 25 to about 45 wt. %, about 25 to about 40 wt. %, about 25 to about 37 wt. %, about 25 to about 34 wt. %, about 25 to about 31 wt. %; from about 28 to about 50 wt. %, about 28 to about 45 wt. %, about 28 to about 40 wt. %, about 28 to about 37 wt. %, about 28 to about 34 wt. %, about 28 to about 31 wt. %; from about 31 to about 50 wt. %, about 31 to about 45 wt. %, about 31 to about 40 wt. %, about 31 to about 37 wt. %; from about 34 to about 50 wt. %, about 34 to about 45 wt. %, about 34 to about 40 wt. %; from about 37 to about 50 wt. %, about 37 to about 45 wt. %, or any range or subrange thereof, based on the total weight of the oral care composition.

In some embodiments, the oral care composition preferably is substantially free or free of caloric sweeteners. For example, the oral care compositions may have about 4 wt. % or less, about 3 wt. % or less, about 2 wt. % or less, about 1 wt. % or less, about 0.5 wt. % or less, or about 0.1 wt. % or less of caloric sweeteners, based on the weight of the oral care composition. In at least one embodiment, the oral care composition contains about 0 wt. % or 0 wt. % of caloric sweeteners, based on the weight of the oral care composition.

The oral care compositions may include a fluoride ion source. The fluoride ion source may be present in an effective amount. In some cases, the fluoride ion source is present in the oral care composition in an amount of about 0.01 to about 5 wt. %, based on the total weight of the oral care composition. For example, the fluoride ion source is present in an amount of about 0.01 to about 5 wt. %, about 0.01 to about 4 wt. %, about 0.01 to about 3 wt. %, about 0.01 to about 2 wt. %, about 0.01 to about 1 wt. %; from about 0.05 to about 5 wt. %, about 0.05 to about 4 wt. %, about 0.05 to about 3 wt. %, about 0.05 to about 2 wt. %, about 0.05 to about 1 wt. %; from about 0.1 to about 5 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.

The fluoride ion source may be selected from soluble fluoride ion salts. For example, the fluoride ion source may comprise sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides or a combination of two or more thereof. In some embodiments, the fluoride ion source comprises sodium fluoride, sodium monofluorophosphate, or a combination thereof. Additional examples of fluoride ion source are disclosed in U.S. Pat. Nos. 3,535,421, 4,885,155, and 3,678,154, the disclosure of each of which is hereby incorporated by reference in their entirety.

In certain embodiments, the oral care composition of the disclosure may contain stannous fluoride and a source of fluoride ions or fluorine-providing agents in amounts sufficient to deliver, in total, from 25 ppm to 25,000 ppm (mass fraction) of fluoride ions, generally at least 500 ppm, e.g., from about 500 to about 2000 ppm, from about 800 to about 1800 ppm, from about 1000 to about 1600 ppm, from about 1200 to about 1550 ppm, or about 1450 ppm.

The oral care compositions may include an anti-calculus agent. The amount of anti-calculus agent(s) present in the oral are composition may be from about 0.5 to about 10 wt. %, based on the total weight of the oral care composition. In some instances, the anti-calculus agent(s) is present in the oral care composition in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The anti-calculus agent(s) may be chosen from phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates, and combinations of two or more thereof. Examples of anti-calculus agents include tetrasodium pyrophosphate, ammonium tripolyphosphate, hexametaphosphate salt, polycarboxylate polymers, polyvinyl methyl ether/maleic anhydride (PVME/MA) copolymers, or combinations of two or more thereof. In some embodiments, the oral care composition includes one or more alkali metal polyphosphate, e.g., as tetrasodium pyrophosphate.

The oral care compositions of the present disclosure may include a flavoring agent. The flavoring agent is typically incorporated in the oral care composition at a concentration of about 0.01 to about 3 wt. % by weight of the oral care composition. For example, the amount of flavoring agent(s) present in the oral care composition may be from about 0.01 to about 2 wt. %, about 0.01 to about 1 wt. %, about 0.01 to about 0.5 wt. %, about 0.01 to about 0.1 wt. %; from about 0.1 to about 3 wt. %, about 0.1 to about 2 wt. %, about 0.1 to about 1 wt. %, about 0.1 to about 0.5 wt. %; from about 0.5 to about 3 wt. %, about 0.5 to about 2 wt. %, about 0.5 to about 1 wt. %; from about 1 to about 3 wt. %, about 1 to about 2 wt. %; from about 2 to about 3 wt. %, including any range or subrange thereof, based on the total weight of the oral care composition.

Suitable flavoring agents include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Additional flavoring agents may include, but are not limited to menthol, artificial vanilla, cinnamon derivatives, and various fruit flavors, spearmint oil, peppermint oil, cinnamon oil, oil of wintergreen (methylsalicylate), clove oil, bay oil, anise oil, eucalyptus oil, thyme oil, cedar leaf oil, oil of nutmeg, oil of sage, oil of bitter almonds, cassia oil, and a combination of two or more thereof.

The oral care compositions may include one or more colorants. Exemplary colorants can include natural or uncertified colors from natural sources or certified colors for the effect of color. In some embodiments, the colorant can include dyes, certified aluminum lakes or colors derived from a natural source. The colorant may be water-based, oil-based or dry. The colorants can be primary colors, blends of colors or discrete mixtures of colors, such as confetti. The concentrations of the colorant in the oral care composition may be from trace amount to about 0.6 wt. %, from about 0.1 to about 0.5 wt. %, about 0.2 to about 0.4 wt. %, or about 0.15 to about 0.35 wt. %, based on the total weight of the oral care composition.

The oral care composition may include one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the oral care composition.

The oral care compositions may have a pH from 4.5 to about 10, 4.5 to about 9, 4.5 to about 8, 4.5 to about 7, 4.5 to about 6; from about 5 to about 10, about 5 to about 9, about 5 to about 8, about 5 to about 7, about 5 to about 6; from about 6 to about 10, about 6 to about 9, about 6 to about 8, or about 6 to about 7; from about 7 to about 10, about 7 to about 9, or about 7 to about 8, including any ranges and subranges therebetween.

### EXAMPLES

### Example 1

Two non-limiting, example oral care compositions (Example Compositions A and B) were prepared in the form of dentifrices in accordance with aspects of the invention. A comparative oral care composition (Comparative Composition 1) was also prepared in the form of a dentifrice. The formulations for Example Compositions A and B and Comparative Composition 1 are provided in Table 1 (below).

TABLE 1

| | Comp. 1 | Ex. A | Ex. B |
|---|---|---|---|
| US INCI Name | (wt. %) | (wt. %) | (wt. %) |
| Sorbitol | 21 | 21 | 21 |
| Glycerin | 10 | 10 | 10 |
| High Cleaning Silica | 20 | 20 | 20 |
| Carbomer 974P | 0 | 1 | 0.5 |
| Sodium Hydroxide | 0 | 0.4 | 0.2 |
| Sodium Lauryl Sulfate | 1.5 | 1.5 | 1.5 |
| Tetrasodium | 0.5 | 0.5 | 0.5 |
| Pyrophosphate | | | |
| Thickening Silica | 4 | 4 | 4 |
| Xanthan Gum | 0.8 | 0 | 0.4 |
| Sodium Fluoride | 0.24 | 0.24 | 0.24 |
| Sodium Saccharin | 0.3 | 0.3 | 0.3 |
| Flavor | 1.2 | 1.2 | 1.2 |
| Water | QS to | QS to | QS to |
| | 100 | 100 | 100 |

### Example 2

Example Composition A was evaluated in comparison to Comparative Composition 1 using an in-vitro brushing study of stained bovine teeth. Artificially stained bovine teeth were obtained from Therametric Technologies, Inc™. The artificially stained bovine teeth were 10 mm² blocks mounted in a methacrylate resin so that only the enamel was exposed to the stain. The staining procedure included cycling the bovine teeth between air and a staining broth containing coffee, tea, mucin, soy broth, and Sarcina Lutea in an incubator at a temperature of 37° C. until the L* value of the bovine teeth is between 25 and 40.1.

The bovine teeth used in this Example were selected following a 20 min brushing using 250 grams of force and a toothpaste slurry consisting of 1 part Max Fresh toothpaste to 1 part water. The bovine teeth were then evaluated using a Spectroshade Micro™ obtained from Medical High Technology (MHT) instrument to determine the initial whiteness level of the bovine teeth. To measure the initial whiteness level, the instrument was positioned so that one tooth was in the instrument's field of vision and then the image was captured. Bovine teeth having an L* value near 60 after brushing were selected for this study. Specifically, 8 bovine teeth were selected for evaluating each of Example Composition A and Comparative Composition 1.

A dentifrice slurry was then prepared by combining 10 grams of water and 10 grams of one of the respective dentifrice compositions. The dentifrice slurry was mixed by hand to completely homogenize the solution.

The bovine teeth were then brushed using brushing machine. Specifically, the heads of soft toothbrushes were cut from their handles and mounted for use on the brushing machine. Samples of the respective dentifrice slurries were poured over trays and brushing was immediately started. The bovine teeth were brushed for 2 minutes (min) using 250 grams of pressure. The brushing machine was set to 120 strokes/min. After 2 minutes, the brushing was stopped, the dentifrice slurry was removed, and the bovine teeth were rinsed with 100 grams of deionized water. The brushing treatment was repeated a total of 14 times to model twice daily use of each product for 7 days.

After the brushing treatment, the bovine teeth were evaluated to determine their final whiteness level using a Spectroshade Micro™ instrument. The procedure for evaluating the final whiteness level was substantially similar to the procedure for evaluating the initial whiteness level. Software from MHT was used to measure the L*, a*, and b* values for each tooth. L* represents lightness from black to white on a scale of zero to 100, while a* and b* represent chromaticity with no specific numeric limits. Negative a* corresponds with green, positive a* corresponds with red, negative b* corresponds with blue and positive b* corresponds with yellow. Using the software, the image of the tooth after treatment was superimposed on the baseline tooth image, the exact same area selected, and the L*, a*, and b* values for the tooth before and after treatment captured. The L*, a*, and b* values were used to calculate the change in the whiteness index for each tooth after brushing treatments as compared to initial whiteness level before the brushing treatment. The whiteness index is calculated as ΔW*, where

$$W * {( a {*}^{2} + b {*}^{2} + {( L * - 100 )}^{2} )}^{1 / 2}$$

$$Δ W *= W {*}_{final} - W {*}_{initial}$$

It should be noted that the more negative the value of ΔW*, the closer the tooth color is to white. A summary of the whiteness index for Example Composition A and Comparative Composition 1 is shown in Table 2 (below).

TABLE 2

| | Comp. 1 | Ex. A |
|---|---|---|
| ΔW* (W*_{Final} − W*_{Initial}) | −2.7 ± 1.0 | −5.3 ± 0.9 |

### Example 3

Example Composition A was evaluated in comparison to Comparative Composition 1 using HAP disks to assess the stain resistance of Example Composition A. To determine the initial color of the HAP disks, each of the HAP disks was placed in well plates and soaked in 4 mL of water and then incubated for an hour. The HAP disks were removed from the well plates, pat dried, and evaluated using a Spectroshade Micro™ instrument to determine the L, a, and b values of each disk. The procedure for evaluating the whiteness level of the HAP disks was similar to the procedures described in Example 1.

A dentifrice slurry sample was then applied to the HAP disks. The dentifrice slurry samples were prepared by combining a sample of either Example Composition A or Comparative Composition 1 with water in amounts to produce dentifrice slurry samples having a weight ratio of the respective dentifrice to water of 1:2. The HAP disks were then placed in separate well plates and 4 mL of respective dentifrice slurry samples was added thereto for 2 minutes. The HAP disks were then removed from the well plates and rinsed with 5 mL of water three times.

The HAP disks were then stained with a coffee solution, which was prepared by dissolving 2 grams of coffee for every 98 grams of water. Specifically, the HAP disks were placed in separate well plates and 4 mL of the coffee solution was added thereto. The HAP disks were kept in the coffee solution for 90 minutes. The HAP disks were then removed from the well plates and rinsed with 5 mL of water three times. The HAP disks were pat dried and then immediately evaluated to assess the final color of the HAP disks using a Spectroshade Micro™ instrument to determine the final L, a, and b values of the HAP disks. A summary of the change in color or whiteness (ΔW*) for Example Composition A and Comparative Composition 1 is shown in Table 3.

TABLE 3

| | Comp. 1 | Ex. A |
|---|---|---|
| ΔW* (W*_{Final} − W*_{Initial}) | 23.6 ± 1.8 | 16.7 ± 1.6 |

What is claimed is:

1. An oral care composition comprising:
a thickening system comprising:
a cross-linked polyacrylic acid and/or a salt thereof, and
one or more thickening agent selected from a polysaccharide, a thickening silica, and a combination of two or more thereof; and
from about 14 to about 64 wt. % of water, wherein all weight percentages are based on the total weight of the oral care composition.

2. The oral care composition according to claim 1, wherein the cross-linked polyacrylic acid and/or the salt thereof is a branched polyacrylic acid and/or a salt thereof.

3. The oral care composition according to claim 1, wherein the cross-linked polyacrylic acid and/or the salt thereof comprises a carbomer.

4. The oral care composition according to claim 1, wherein the cross-linked polyacrylic acid and/or the salt thereof consist of one or more carbomer.

5. The oral care composition according to claim 1, wherein the cross-linked polyacrylic acid and/or the salt thereof is present in an amount from about 0.2 to about 5 wt. %.

6. The oral care composition according to claim 1, further comprising a polyol system present in a total amount of about 20 to about 40 wt. %, wherein the polyol system comprises at least two polyols selected from glycerin, inositol, maltitol, mannitol, sorbitol, xylitol, propylene glycol, polypropylene glycol (PPG), polyethylene glycol (PEG), a block copolymer of PPG and PEG, a saccharide, and a combination of two or more thereof.

7. The oral care composition according to claim 6, wherein the polyol system comprises glycerin and sorbitol present in a weight ratio of the glycerin to the sorbitol of about 1:1 to about 3:1.

8. The oral care composition according to claim 1, further comprising an abrasive in an amount from about 1 to about 35 wt. %, wherein the abrasive is selected from the group consisting of silica, silicate, silicon, bentonite, zeolite, kaolin, mica, pumice, calcium carbonate, cuttlebone, insoluble phosphates, polycarbonate, microcrystalline wax, microcrystalline cellulose, and a combination of two or more thereof.

9. The oral care composition according to claim 1, wherein the water is present in an amount from about 30 to about 50 wt. %.

10. The oral care composition according to claim 1, further comprising a fluoride source in an amount from about 0.1 to about 7 wt. %, wherein the fluoride source is selected from stannous fluoride, sodium fluoride, potassium fluoride, sodium monofluorophosphate, sodium fluorosilicate, ammonium fluorosilicate, amine fluoride, ammonium fluoride, and a combination of two or more thereof.

11. The oral care composition according to claim 1, further comprising an anti-calculus agent in an amount from about 0.5 to about 10 wt. %, wherein the anti-calculus agent is selected from tetrasodium pyrophosphate, ammonium tripolyphosphate, hexametaphosphate salt, polycarboxylate polymers, polyvinyl methyl ether/maleic anhydride copolymers, and a combination of two or more thereof.

12. The oral care composition according to claim 1, wherein the thickening system is present in an amount about 2 to about 15 wt. %, wherein the thickening system comprises a thickening silica and a polysaccharide.

13. The oral care composition according to claim 12, wherein the polysaccharide is a natural gum selected from alginate, pectin, carrageenan, gum arabic, tragacanth gum, karaya gum, agarose, guar gum, pechtin, gellan gum, tara gum, locust bean gum, tamarind gum, locust bean gum, Seneca gum, sclerotium gum, gellan gum, xanthan gum, and a combination of two or more thereof.

14. The oral care composition according to claim 1, wherein the oral care composition is substantially free of or free of polyvinyl thickeners.

15. The oral care composition according to claim 1, wherein the oral care composition is substantially free of or free of peroxides.

16. The oral care composition according to claim 1, wherein the oral care composition is substantially free of or free of whitening agents.

17. An oral care composition comprising:
a thickening system comprising:
from about 0.2 to about 5 wt. % of a cross-linked polyacrylic acid and/or a salt thereof, and
one or more thickening agent selected from a polysaccharide, a thickening silica, and a combination of two or more thereof;
a polyol system comprising glycerin and sorbitol present in a weight ratio of glycerin to sorbitol of about 1:1 to about 3:1; and
from about 5 to about 64 wt. % of water,
wherein the oral care composition is free of whitening agents, and all weight percentages are based on the total weight of the oral care composition.

18. The oral care composition according to claim 17, wherein the cross-linked polyacrylic acid and/or a salt thereof is present in an amount from about 0.5 to about 4 wt. %.

19. The oral care composition according to claim 17, wherein the oral care composition is substantially free or free of calcium carbonate.

20. The oral care composition according to claim 17, wherein the oral care composition has a relative dental abrasivity of about 10 to about 250, as determined pursuant to standards DIN EN ISO 11609.
